(19) United States
(12) Patent Application Publication
(10) Pub. No.: US 2025/0257888 A1
(43) Pub. Date: Aug. 14, 2025

(54) A LIGHT BASED DISINFECTION DEVICE

(71) Applicant: SIGNIFY HOLDING B.V., EINDHOVEN (NL)

(72) Inventors: MARC ANDRE DE SAMBER, LOMMEL (BE); EVREN ÖZCAN, EINDHOVEN (NL)

(21) Appl. No.: 18/857,889

(22) PCT Filed: Apr. 12, 2023
(86) PCT No.: PCT/EP2023/059553

§ 371 Date: Oct. 18, 2024

(30) Foreign Application Priority Data
Apr. 19, 2022 (EP) 22168741.1

(51) Int. Cl. F24F 8/22; A61L 9/20

(52) U.S. Cl. CPC F24F 8/22; A61L 9/20; A61L 2209/11; A61L 2209/12

(57) ABSTRACT

The invention provides a disinfection device (100) for disinfecting an air volume (2) of a space (1), wherein the disinfection device (100) comprises: a plenum (10) for retaining the air volume (2); a light source (11) for illuminating the plenum (10); a flow arrangement (101); a controller (14); wherein the controller (14) is configured to control, for each cycle of a plurality of repetitive cycles, the flow arrangement (101) to: (i) force the air volume (2) into the plenum (10), (ii) retain the air volume (2) in the plenum (10) for a retainment period, (iii) release the air volume (2) from the plenum (10) at the end of said retainment period; wherein the controller (14) is configured to control the light source (11) to emit a disinfecting light (19) during the retainment period of each respective cycle, so as to disinfect the air volume (2) retained in the plenum (10).

### FIELD OF THE INVENTION

The invention relates to a (light-based) disinfection device for disinfecting an air volume of a space. The invention further relates to a corresponding method of disinfection.

### BACKGROUND OF THE INVENTION

Societal health is periodically contested by virus outbreaks, such as seasonal symptomatic influenza A/B outbreak, SARS, MERS, COVID-19. Future outbreaks, mutations, epidemics, and pandemics are not excluded. At least partly due to an increasing population, urbanization and people movement, the topic of people health & wellbeing is becoming more and more relevant.

Such developments have clearly risen the demand for various disinfection devices to disinfect air from pathogens. For example, a clear demand exists for light-based disinfection devices. Ultraviolet light-based disinfection devices are particularly employed due to their strong germicidal effect.

However, since various spectra of disinfecting ultraviolet light may also be hazardous to humans and animals, in certain doses and time of exposures, such ultraviolet light-based disinfection devices may be perceived as dangerous, particularly for disinfection devices that directly illuminate the ambient. Therefore, ultraviolet light-based disinfection devices, which disinfect the air internally, may be preferred for disinfecting spaces occupied by humans and/or animals. For example, the Philips UVCA200 UV-C Floor standing air unit.

Such internally operated ultraviolet light-based disinfection devices typically generate a continuous airflow through device. Contaminated air is thereby drawn in, guided subsequently along an ultraviolet light source to get disinfected, and the disinfected air is finally blown out. Effectiveness of disinfection may be achieved by displacing large volumes of air in a short moment of time and illuminating said flowing air with significant doses of ultraviolet light.

However, such operation may require high intensities of ultraviolet light for effective disinfection; particularly when it is needed to bring the concentration of an active or infectious airborne pathogen below a certain health threshold level with a single passage of the volume of air carrying an airborne pathogen through such a disinfection device. Therefore, internally operated ultraviolet light-based disinfection devices may not be power efficient.

Another consequence is that the size of the apertures for sucking in the contaminated air, and blowing out the disinfected air, may scale with the volume flow rate of the air put through the device for disinfection. Hence, a larger inlet and outlet aperture may be desired, for higher volume flow rates handled by the device. Such larger dimensions may however increase the risk of ultraviolet light leakage to the exterior of the disinfection device; or may impose the need for even more stringent safety measures, or design constraints (such as chicanes to block UV-C light).

Hence, a clear need exists to improve the operation of such internally operated (ultraviolet) light-based disinfection devices; amongst others to improve safety and the power consumption performance thereof, while still maintaining the desired disinfection effectiveness.

### SUMMARY OF THE INVENTION

It is an object of the invention to provide an improved disinfection device for disinfecting an air volume of a space, which at least alleviates the problems and disadvantages mentioned above, and renders a more efficient solution. Thereto, the invention provides a disinfection device for disinfecting an air volume of a space, wherein the disinfection device comprises: a plenum for retaining the air volume; a light source for illuminating the plenum; a flow arrangement; a controller; wherein the controller is configured to control, for each cycle of a plurality of repetitive cycles, the flow arrangement to: (i) force the air volume into the plenum, (ii) retain the air volume in the plenum for a retainment period, (iii) release the air volume from the plenum at the end of said retainment period; wherein the controller is configured to control the light source to emit a disinfecting light during the retainment period of each respective cycle, so as to disinfect the air volume retained in the plenum; wherein the emitted disinfecting light comprises a light intensity; wherein the controller is configured to set said light intensity based on the duration of said retainment period; wherein the controller is configured to determine said retainment period based on a predetermined retainment period stored within said controller; OR receive an input indicative of said retainment period, wherein the controller is configured to determine said retainment period based on the received input.

Hence, the present invention provides a disinfection device comprising a plenum and flow arrangement. The flow arrangement is arranged for controlling (or: managing, or: facilitating) the flow of the respective air volume through the disinfection device. The plenum is configured to retain an air volume of a space. The plenum may alternatively be phrased as an air reservoir.

Moreover, the present invention provides a disinfection device that operates in a plurality of cycles. For each cycle of said plurality of cycles, an air volume is forced into the plenum and retained for a retainment period. During this retainment period, the light source disinfects the air volume that is retained in the plenum with said disinfecting light. Consequently, at the end of the retainment period of the respective cycle, the air volume is released from the plenum. Therefore, each cycle of the plurality of cycles, as defined in the present invention, renders a disinfected air volume. Phrased alternatively, said steps (i), (ii), (iii) may be controlled subsequently, or consecutively.

The present invention is advantageous, because the disinfection of the air of the space occurs intermittently in a plurality of cycles, wherein each respective air volume can be disinfected during a particular retainment period with said disinfecting light. As the air volume is retained for the retainment period, and thereby exposed a longer time to the disinfecting light, the light source may more effectively disinfect the air. This improves the disinfection effectiveness of the disinfection device.

Furthermore, since the air volume is retained for the retainment period, and thereby exposed longer to the disinfecting light, the light source may set to provide the disinfecting light at a lower intensity, but still ensure the disinfection effectiveness and a germicidal effect. Thereby, the disinfection device according to the present invention may require less power consumption for disinfection, particularly when the light source emits ultraviolet light, but still maintain a desired disinfection effectiveness (for example compared to light-based disinfection devices in which the air is flowing continuously and at higher flow rate past the disinfecting light source).

Even further, as the air volume is exposed longer to the disinfecting light in the present invention, the light source may similarly provide the disinfecting light at less hazardous wavelengths of light, but still ensure the disinfection effectiveness and germicidal effect. Thereby, the disinfection device according to the present invention may be improved in safety, while still rendering a same disinfection effect. This is supported further by the at least one valve retaining the air volume in the plenum, closing the plenum from fluid communication with the space, and thereby also mitigating a possible risk of hazardous light leakage, which is particularly advantageous when disinfecting with for example certain wavelengths of UVC light.

Hence, the disinfection device according to the invention clearly alleviates the problems and disadvantages mentioned above, and renders an improved light-based disinfection device, particularly when operated with ultraviolet light.

In embodiments, the flow arrangement may comprise a flow generator and/or at least one valve. The flow of the air volume through the disinfection device may utilize known and established concepts in fluid mechanics.

In embodiments, the controller may be configured to control, for each cycle of the plurality of repetitive cycles, the flow generator to force the air volume into the plenum, and/or to release the air volume from the plenum at the end of said retainment period. Said release may cause the air volume to be in fluid communication with the space; and diffuse back into the space after being disinfected.

The release may be facilitated further by the flow generator forcing the air volume out of the plenum. Hence, in an embodiment. The controller may for example be configured to control, for each cycle of the plurality of repetitive cycles: the flow generator to force the air volume out of the plenum at the end of said retainment period.

In embodiments, the flow arrangement comprises at least one valve; wherein the at least one valve may be configured to operate between an open position and a closed position, wherein in the open position the plenum is in fluid communication with the space, wherein in the closed position the plenum is not in fluid communication with the space; wherein the controller may be configured to control, for each cycle of the plurality of repetitive cycles, the at least one valve to retain the air volume in the plenum for the retainment period, and/or to release the air volume from the plenum at the end of said retainment period.

Therefore, the plenum may comprise said at least one valve (of the flow arrangement). The at least one valve may be configured to operate between an open position, in which the plenum is in fluid communication with the space, thereby enabling an air volume to enter or exit the plenum, and a closed position, in which the plenum is not in fluid communication with the space, thereby retaining an air volume within the plenum.

Hence, during each cycle of the plurality of cycles, the controller may control the flow generator (of the flow arrangement) only at the start of the retainment period to force air into the plenum. Since the forcing of the air volume may generate most noise during operation, the present invention also renders a benefit in that disinfection occurs with less noise, or different type of noise, compared to continuously and internally operated light-based disinfection devices.

The at least one valve may retain the air volume in the plenum for the retainment period in the closed position. The at least one valve may release the air volume from the plenum at the end of said retainment period in the open position. The at least one valve may be in the open position when the flow generator forces the air volume into the plenum.

In an embodiment, the at least one valve may be a first valve. The first valve may operate between said open position and said closed position, wherein in the open position the plenum is in fluid communication with the space, wherein in the closed position the plenum is not in fluid communication with the space. The first valve may retain the air volume in the plenum for a retainment period (in the closed position). The first valve may release the air volume from the plenum at the end of said retainment period (in the open position). The first valve may be in the open position when the flow generator forces the air volume into the plenum. Hence, in such an embodiment, the plenum comprises only a first valve, that enables the air volume to enter the plenum, but also to exit the plenum.

In an embodiment, the at least one valve may be a first valve and a second valve. The first valve may operate between said open position and said closed position. The second valve may also operate between said open position and said closed position. As mentioned, in the open position the plenum is in fluid communication with the space, and in the closed position the plenum is not in fluid communication with the space. The first valve and the second valve may both retain the air volume in the plenum for a retainment period (in the closed position). The second valve may release the air volume from the plenum at the end of said retainment period (in the open position). The first valve may be in the closed position when said air volume is released from the plenum at the end of said retainment period. The first valve may be in the open position when the flow generator forces the air volume into the plenum. The second valve may be in the closed position when the flow generator of the flow arrangement, or another fluid flow means, for example, forces the air volume into the plenum.

Hence, in such an embodiment, the plenum comprises a first valve and a second valve, which first valve is operated to the open position to enable the air volume to enter the plenum, while the second valve is operated to the open position to enable the air volume to be released from the plenum. Thus, the air volume enters the plenum via the first valve and releases from the plenum via the second valve. The first valve may be phrased as the inlet valve and the second valve may be phrased as the exit valve.

In aspects, the release of the air volume may be facilitated further when the air volume is retained in a pressurized manner in the plenum, such that when the at least one valve is in the open position, the air volume may be forced back into the space (due to the resulting pressure difference). Hence, in an embodiment, controller may be configured to control, for each cycle of the plurality of repetitive cycles: the flow arrangement, or more specifically said flow generator, to pressurize the air volume in the plenum.

In aspects, the plenum may comprise at least one conduit to the space. In aspects, each conduit of the at least one conduit may comprise a respective valve of the at least one valve. For example, the plenum may comprise a first conduit and a second conduit, and the plenum may comprise a first valve and a second valve, wherein the first valve is arranged in the first conduit, wherein the second valve is arranged in the second conduit.

In aspects, the plenum may comprise a volume between a twentieth of a cubic meter and a cubic meter, preferably between a twentieth of a cubic meter and a quarter of a cubic meter, for example a tenth of a cubic meter.

As partly mentioned, the controller is configured to control, for each cycle of the plurality of repetitive cycles, the flow arrangement to retain the air volume in the plenum for a retainment period, during which the light source emits the disinfecting light and illuminates the plenum, so as to disinfect the air volume retained in the plenum. The retainment period is therefore a relevant parameter in the operation of the disinfection device according to the invention. The retainment period should for example provide sufficient time to expose the air volume to the disinfection light. This may provide an improved disinfection performance, but may for example also enable the light source to be operated at a more power efficient intensity setting, and/or at a less hazardous spectrum, while still maintaining a desired disinfection effectiveness.

Hence, in an embodiment, the retainment period may be at least one minute. For example, the retainment period being at least one minute, may render that at most sixty air volumes may be disinfected in an hour. In aspects, the retainment period may be at least two minutes. In aspects, the retainment period may be at most ten minutes.

In an embodiment, the emitted disinfecting light may comprise a light spectrum; wherein the light spectrum comprises at least one dominant peak wavelength in the ultraviolet wavelength range from 100-380 nm. Hence, the present invention provides light-based disinfection with ultraviolet light.

Hence, the disinfecting light according to the invention may comprise ultraviolet light having an ultraviolet wavelength range. The germicidal effect of ultraviolet light is well known in the art. The ultraviolet wavelength range is defined as light in a wavelength range from 100 to 380 nm. UV light suitable for disinfection purposes may in general terms be divided into three main types, namely UV-A light with a wavelength in the range of 315 to 400 nm, UV-B light with a wavelength in the range of 280 to 315 nm and UV-C light with a wavelength in the range of 100 to 280 nm. UV-C light may further be divided into Near UV-C light with a wavelength in the range of 230-280 nm, Far UV-C light with a wavelength in the range of 190-230 nm, and extreme UV-C light with a wavelength in the range of 100-190 nm.

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may inactivate (kill) bacteria, but may be less effective in inactivating (killing) viruses. UV-B may be (relatively) safe when a low dose (i.e. low exposure time and/or low intensity) is used, may inactivate (kill) bacteria, and may be moderately effective in inactivating (killing) viruses. UV-B may also have the additional benefit that it can be used effectively in the production of vitamin D in a skin of a person or animal. Near UV-C may be relatively unsafe, but may effectively inactivating, especially kill bacteria and viruses. Far UV may also be effective in inactivating (killing) bacteria and viruses, but may be (relatively to other UV-C wavelength ranges) (rather) safe. Far-UV light may generate some ozone which may be harmful for human beings and animals. Extreme UV-C may also be effective in inactivating (killing) bacteria and viruses, but may be relatively unsafe. Extreme UV-C may generate ozone which may be undesired when exposed to human beings or animals. In some application ozone may be desired and may contribute to disinfection, but then its shielding from humans and animals may be desired.

As mentioned, the emitted disinfecting light may comprise a light intensity;

wherein the controller may be configured to set said light intensity based on the duration of said retainment period. For example, the light source may emit the disinfecting light with a lower intensity when the duration of the retainment period becomes larger.

For example, the controller may set the light intensity to at most a halve of the maximum light intensity of the light source for a retainment period of at least two minutes. For example, the controller may set the light intensity to at most a quarter of the maximum light intensity of the light source for a retainment period of at least four minutes. Other examples may be envisioned similarly. The required exposure time to UV-C light for a certain log-reduction of a known pathogen may for example be found in literature, such as the Ultraviolet Germicidal Irradiation Handbook of Kowalski, ISBN 978-3-642-01998-2.

In an embodiment, the emitted disinfecting light may comprise a light spectrum; wherein the controller may be configured to set said light spectrum based on the duration of said retainment period. For example, the light source may emit the disinfecting light at a light spectrum comprising less UV-C wavelengths and/or more UV-A wavelength when the duration of the retainment period becomes larger.

In an embodiment, the light source may be at least one LED light source. For example, the light source may be a plurality of LED light sources. In aspects, the light source may be at least one semiconductor lighting device. The light source may be at least one conventional light source, such as a fluorescent tube, batten, or bulb. The light source may be a laser light source. The light source may be a LED strip. The light source may be a LED panel.

In an embodiment, the light source may be a Xenon lamp. Said Xenon lamp may for example emit in operation ultraviolet light, such as Far UV-C light. High intensity pulsed Xenon lamp light may also render a germicidal effect, without having a long exposure time.

In an embodiment, the controller may be configured to control light source to emit at least one pulse of disinfecting light during the retainment period of each respective cycle, so as to disinfect the air volume retained in the plenum; wherein each respective pulse of the at least one pulse comprises a time duration between one microsecond and ten milliseconds. For example, considering this embodiment, said light source may preferably be a Xenon lamp. Such an embodiment may be advantageous, as a pulsed light emittance reduces the exposure of possibly harmful disinfecting light, and may improve safety in the space. Said at least one pulse may for example be at most 100 pulses during the retainment period, at most 20 pulses during the retainment period, or for example a single pulse during the retainment period.

In further aspects, the light source may comprise both a conventional light source, such as e.g. a Xenon lamp, and a semiconductor light source, such as a LED light source. Said Xenon lamp and said LED light source may both for example emit at least partly in the UV spectrum.

The disinfecting device according to the invention may also disinfect in steps. During a first step of disinfection with light, the air volume may be irradiated with a first disinfecting light that targets the primarily proteins and non-nucleic acid macromolecules of pathogens, such as for example far UVC light (e.g., 222 nm, 210 nm). This may break and/or disrupt elements of the chemical groups of the pathogen (to be disinfected) and/or the chemical compounds in the particles that contain the pathogen (to be disinfected). This results in that the ‘inside’ of the pathogen (or the inside of the particle/droplet carrying the pathogen) opens up for irradiation with longer wavelengths.

Hence, during the second step of disinfection with light, the air volume (that has been subject to the first step) may be irradiated subsequently with a second (and different) disinfecting light. The second disinfecting light may for example be more harmful to humans relative to the first disinfecting light, such as 254 nm UV-C light relative to 222 nm Far UV-C light. Because the first disinfecting light has already made the pathogen more susceptible to disinfection with less harmful light, the (more harmful) second disinfecting light does not require a higher dose to achieve a certain disinfection level as compared to a situation without the first disinfecting light being applied. Hence, the second disinfecting light is arranged to easier kill the pathogen, because the second disinfecting light can now easier penetrate in the pathogen (e.g. virus) and/or easier penetrate into the droplet/particle (e.g. aerosol). Hence, the efficiency of RNA or DNA genetic damaging (dimerization) of the pathogen will increase, resulting in a better reduction of multiplication potential of the pathogen.

Hence, in an embodiment, the disinfecting light may comprise a first disinfecting light and a different second disinfecting light; wherein the controller is configured to control the light source to emit the first disinfecting light for a first subperiod during the retainment period of each respective cycle, and the second disinfecting light for a second subperiod during the retainment period of each respective cycle, so as to disinfect the air volume retained in the plenum; wherein the first subperiod precedes the second subperiod.

In an embodiment thereof, the first disinfecting light may for example comprise Far-UVC light; wherein the second disinfecting light may for example comprise Near-UVC light, preferably 254 nm Near-UVC light.

In an embodiment, the plenum may comprise a plenum wall enclosing the plenum; wherein the plenum wall may comprise a reflective surface facing into the plenum, wherein the reflective surface may be configured to reflect the disinfecting light. Such a reflective surface may improve the disinfection performance of the disinfection device, as the air volume is retained within the plenum, also the disinfection light is reflected back, and thereby kept within the plenum. In aspects, the reflective surface may be a retroreflector. In aspects, the reflective surface may be a reflective surface coating.

In an embodiment, the plenum may comprise a plenum wall enclosing the plenum; wherein the plenum wall may comprise a light guide for conveying light into the plenum; wherein the light source may be configured to couple light into said light guide for illuminating the plenum. Such a light guide may be advantageous, because the light source may be external to the plenum for coupling in the disinfecting light into the light guide, and/or the disinfecting light may be more evenly distributed within the plenum.

In an embodiment, the light source may be arranged inside the plenum. Such an embodiment may be advantageous as the light source may be directly within the plenum comprising the air volume. For example, the light source may be arranged on an inner surface of a plenum wall of the plenum.

In an embodiment, the plenum may comprise an upper halve region and a nonoverlapping lower halve region, the upper halve region and the lower halve region comprising a same volume value, wherein the lower volume region is located below the upper volume region with respect to the gravitational direction; wherein the light source may be arranged inside the plenum within the lower volume region. By having the light source arranged inside the plenum within the lower volume region, the heat generated by the light source emitting the disinfecting light (which may be considerable for e.g. ultraviolet light) may heat the air volume in said lower volume region. Buoyancy effects may subsequently render natural convection within the plenum, thereby mixing the air volume, and improving disinfection performance.

In an embodiment, the light source may be arranged outside the plenum; wherein the plenum is at least partly transparent for the disinfecting light. Such embodiments may facilitate the production of the disinfection device, as the light source does not have to be mounted inside the plenum. For example, the light source may be arranged outside the plenum, and at least partly surround the plenum.

In aspects, the plenum may comprise a plenum material, wherein said plenum material may for example be a transparent polymer, transparent silica glass, transparent quartz, transparent ceramic, or glass.

As mentioned, the controller may be configured to determine said retainment period based on a predetermined retainment period stored within said controller. Hence, the controller already stores the predetermined retainment period, and operates with said predetermined retainment period as a default setting. The controller may for example optionally comprise a local memory for storing said predetermined retainment period.

In aspects, the stored retainment period may be based on a light source characteristic, such as e.g. a (fixed or tunable) energy of the light source, and/or on the variable susceptibility of a certain airborne pathogen. The controller may thus initially set and store the retainment period according to the type of a pathogen (e.g., COVID-19 seasonality, influenza seasonality, etc.).

In an embodiment, the controller may be configured to receive an input indicative of said retainment period, wherein the controller is configured to determine said retainment period based on the received input. Hence the controller may determine said retainment period by considering the received input indicative of said retainment period. Said input may for example be a user input. For example, a user may provide the disinfection device with a user input (or user control input) setting the retainment period. In aspects, the controller may further be configured to determine said retainment period based on the received input and/or a predefined data indicative of the light source according to the invention.

In aspects: the received input indicative of the retainment period may be a pathogen indicator, such that the controller may determine said retainment period based on the pathogen indicator, wherein the pathogen indicator may indicate a type of pathogen (e.g. seasonal variants of: COVID, Influenza, Flu.).

Another input to the controller, via a user interface (e.g. a button, or UI element) comprised by the controller and/or the disinfection device itself, could be the type of pathogen or the actual epidemiological status. For example, at the beginning of the influenza seasons, the apparatus might be set to ‘influenza modus’.

As mentioned, the flow arrangement may comprise a flow generator. In aspects, the flow generator according to the invention may be a fan. In aspects, the flow generator according to the invention may be a bellows. In aspects, the flow generator may force the air into the plenum with a flowrate between 0.005-1 m³/sec, preferably between 0.005-0.5 m³/sec, more preferably between 0.005-0.2 m³/sec.

In aspects, the plenum may comprise an outlet conduit arranged for releasing the air volume to the space. A valve of the at least one valve may be arranged in said outlet conduit. Said outlet conduit may be circular. Said outlet conduit may comprise a nozzle configured to guide (or: direct) the released air volume, when released to the space, into a plug flow, a jet flow, or vortex flow. The disinfection device comprising an outlet conduit with a nozzle configured to release the air volume in a vortex flow may for example be advantageous to distribute disinfected air better in the space. The disinfection device comprising an outlet conduit with a nozzle configured to release the air volume in a plug flow or jet flow may for example be advantageous to distribute disinfected air further (in distance) in the space.

It is further an object of the invention to provide a method of disinfection. Thereto, the invention provides a method of disinfecting an air volume of a space, wherein the method comprises, for each cycle of a plurality of repetitive cycles: forcing the air volume of the space into a plenum; retaining the air volume in the plenum for a retainment period; controlling a light source to emit a disinfecting light during the retainment period, so as to disinfect the air volume retained in the plenum; releasing the air volume from the plenum at the end of said retainment period. Thereby, advantages and/or embodiments applying to the disinfection device according to the invention may mutatis mutandis apply to said method according to the invention. Said method may for example be performed by the disinfection device according to the invention.

In aspects, the invention may provide a disinfection device for disinfecting an air volume of a space, wherein the disinfection device comprises: a plenum for retaining the air volume; a light source for illuminating the plenum; a flow generator; a controller; wherein the plenum comprises at least one valve configured to operate between an open position and a closed position, wherein in the open position the plenum is in fluid communication with the space, wherein in the closed position the plenum is not in fluid communication with the space; wherein the controller is configured to control, for each cycle of a plurality of repetitive cycles: (i) the flow generator to force the air volume into the plenum, (ii) the at least one valve to retain the air volume in the plenum for a retainment period, (iii) the at least one valve to release the air volume from the plenum at the end of said retainment period; wherein the controller is configured to control the light source to emit a disinfecting light during the retainment period of each respective cycle, so as to disinfect the air volume retained in the plenum.

In aspects, the invention may provide, a disinfection device for disinfecting an air volume of a space, wherein the disinfection device comprises: a plenum for retaining the air volume; a light source for illuminating the plenum; a flow generator; a controller; wherein the plenum comprises a first conduit connecting the plenum to the space, a second conduit connecting the plenum to the space, a first valve configured to operate between an open position and a closed position, a second valve configured to operate between an open position and a closed position, wherein in the open position the plenum is in fluid communication with the space, wherein in the closed position the plenum is not in fluid communication with the space; wherein the controller is configured to control, for each cycle of a plurality of repetitive cycles: (i) the flow generator to force the air volume into the plenum via said first conduit, (ii) the first valve and the second valve to retain the air volume in the plenum for a retainment period, (iii) the second valve to release the air volume from the plenum at the end of said retainment period via said second conduit; wherein the controller is configured to control the light source to emit a disinfecting light during the retainment period of each respective cycle, so as to disinfect the air volume retained in the plenum.

### BRIEF DESCRIPTION OF THE DRAWINGS

The invention will now be further elucidated by means of the schematic non-limiting drawings:

FIGS. 1A and 1B depict schematically an embodiment of a disinfection device according to the invention;

FIGS. 2A and 2B depict schematically an embodiment of a disinfection device according to the invention;

FIGS. 3A and 3B depict schematically an embodiment of a disinfection device according to the invention;

FIG. 4 depicts schematically a method according to the invention.

### DETAILED DESCRIPTION OF THE EMBODIMENTS

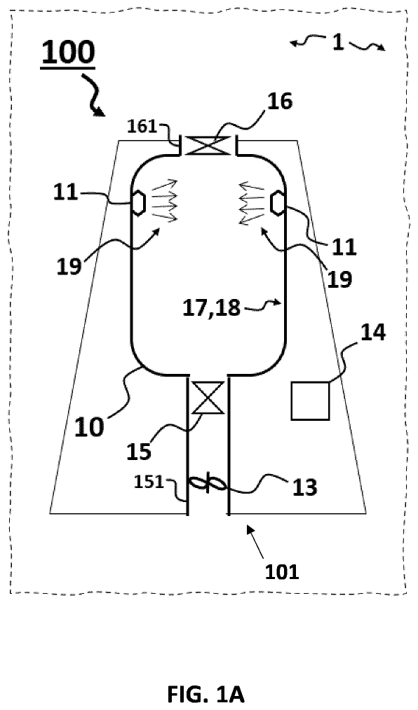

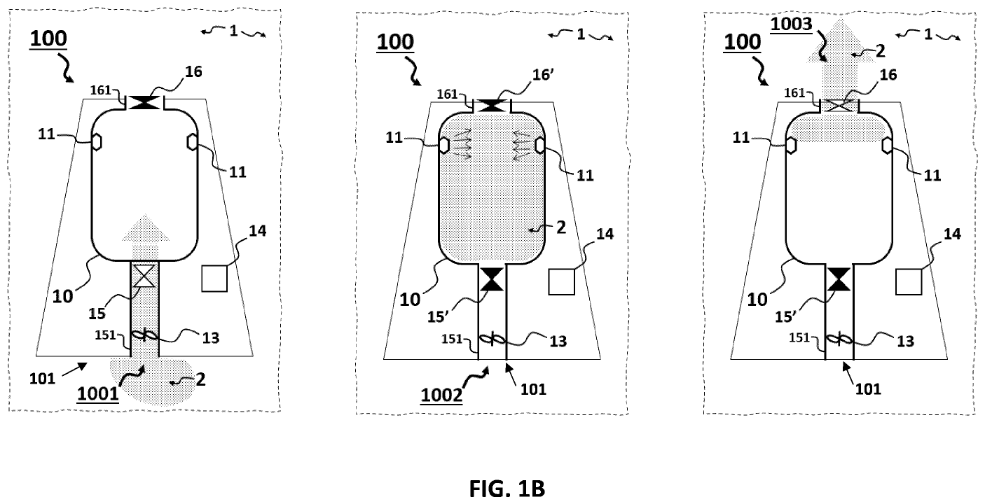

FIG. 1A depicts schematically, by non-limiting example, an embodiment of a disinfection device 100 according to the invention. FIG. 1B depicts schematically, by non-limiting example, the operation of said disinfection device 100.

Referring to FIG. 1A and FIG. 1B, the disinfection device 100 according to the invention is located within a space 1. The space 1 contains air to be disinfected. Said space 1 may for example be an office, a hospital room, a house, a vehicle, etc. The disinfection device 100 according to the invention is configured to operate in a plurality of cycles. For each cycle of said plurality of cycles, a respective air volume 2 of said space 1 is disinfected. Thus, the air volume 2 may be a part of the atmosphere of said space 1.

The disinfection device 100 comprises a plenum 10. The plenum 10 is configured to retain the respective air volume 2 to be disinfected. The disinfection device 100 further comprises a flow arrangement 101. The flow arrangement 101 is configured to control the flow of a respective air volume 2 of the space 1 through the disinfection device 100. Here, by non-limiting example, the flow arrangement comprises a flow generator 13, and at least one valve 15, 16. Such valves may for example be optional. Such valves may alternatively be chicanes (or: chicane conduits) of the plenum. More specifically, the at least one valve 15, 16 of the flow arrangement 101 is embodied as part of the plenum 10.

Hence, the plenum 10 has at least one valve 15, 16. Here, the plenum 10 has a first valve 15 and a second valve 16. The plenum 10 optionally comprises a first conduit 151 to the space 1, and a second conduit 161 to the space 1, wherein the first valve 15 is arranged in the first conduit 151 and the second valve 16 is arranged in the second conduit 161. Alternatively, the plenum may be envisioned to comprise a plurality of valves, and/or a plurality of corresponding conduits.

More specifically, the at least one valve 15, 16 is configured to operate between an open position and a closed position. In the open position, the respective valve of the at least one valve is in fluid communication with the space 1. In the closed position, the respective valve of the at least one valve 15, 16 is not in fluid communication with the space 1. Hence, each respective valve 15, 16 is configured to open or close their corresponding conduit 151, 161, to thereby allow or prevent airflow between the plenum 10 and the space 1. The closed position of a valve is referenced in the figures with the reference numeral having an accent (′).

Still referring to FIG. 1A and FIG. 1B, the disinfection device 100 according to the invention comprises a light source 11. The light source 11 may be a plurality of light sources. Here, the light source 11 are two LED light sources. Here, said LED light sources are ultraviolet LED light sources. The light source 11 is therefore emitting, in operation, disinfecting light 19. The disinfecting light 19 is illuminating the plenum 10 and any respective air volume retained therein.

Here, the disinfecting light 19 comprises a light spectrum comprising at least one dominant peak wavelength in the ultraviolet wavelength range from 100-380 nm. Hence, in other words, the disinfecting light 19 may be ultraviolet light. For example, said disinfecting light may comprise UV-C, Far-UVC, Near-UVC, 354 nm UVC light, 254 nm UVC light, 222 nm UVC light, UV-A, and/or UV-B light, or Xenon light spectrum.

Hence, in alternative embodiments, the light source may be a Xenon lamp. The disinfecting light 19 may alternatively be Xenon light, that is e.g. comprising a peak wavelength in the ultraviolet light spectrum. Even further, the light source, here the Xenon lamp, may be controlled to emit at least one pulse of disinfecting light during the retainment period of each respective cycle, so as to disinfect the air volume retained in the plenum. Each respective pulse of the at least one pulse comprises a time duration between one microsecond and ten milliseconds.

Alternatively, the disinfection device may disinfect said air volume based on for example infrared light or violet light, wherein the disinfecting light may for example comprise a spectrum with a dominant peak in the infrared or violet spectrum.

Furthermore, the light source 11 is arranged inside the plenum 10. More specifically, the plenum 10 comprises a plenum wall 17. The plenum wall 17 encloses the plenum 10. The light source 11 (i.e. the two ultraviolet LED light sources) is connected/mounted to the plenum wall 17 on a surface 18 facing into (the interior of) the plenum 10. Optionally, said surface 18 is reflective to the disinfecting light 19, or said surface may comprise a layer reflective to the disinfecting light 19. Said layer may alternatively be a retroreflector. Having a reflective surface 18 prevents loss of the disinfection light 19 and improves lighting efficiency.

Still referring to FIG. 1A and FIG. 1B, as partly mentioned, the disinfection device 100 further comprises a flow generator 13 of the flow arrangement 101. Here, the flow generator 13 is a fan, or in other words, is a ventilator. Here, the flow generator 13 is consequently arranged in the first conduit 151. Alternatively, said flow generator may e.g. be a bellows. The flow generator 13 is configured to force, in operation, an air volume 2 into the plenum 10, and/or to release the air volume 2 from the plenum 10.

Still referring to FIG. 1A and FIG. 1B, the disinfection device 100 further comprises controller 14. The controller 14 is configured to determine a retainment period. The controller 14 is configured to the disinfection device 100 for a plurality of repetitive cycles. The controller 14 is configured, for each cycle of said plurality of repetitive cycles, to at least perform the below actions (i), (ii), and (iii).

Namely, for said respective cycle, (i) the controller 14 is configured to control the flow generator 13 of the flow arrangement 101 to force an air volume 2 into the plenum 10. The air volume 2 is a part of the atmosphere of the space 1. Hence, the air volume 2 is drawn in, by the flow generator 13, through the first conduit 151, and through the first valve 15 into the plenum 10. The first valve 15 is thereby automatically operated, as the first valve 15 allows the air volume 2 to flow into the plenum 10 but not in the opposite direction out. The flow generator 13 forcing the air volume 2 into the plenum is depicted in FIG. 1B and referenced with reference 1001. The second valve 16 is controlled to remain closed during the flow generator 13 forcing the air volume 2 into the plenum 10.

For said respective cycle, (ii) the controller 14 is also configured to control the at least one valve 15, 16 to retain the air volume 2 in the plenum 10 for the retainment period. This is not necessary, as in other examples only the operation of the flow generator (i.e. the fan or ventilator) may also substantially induce flow and maintain flow in the plenum 10. More specifically, the controller controls the first valve 15 in the closed position (i.e. by not forcing the air volume with the flow generator 13) and the second valve 16 to remain in the closed position. This retaining is depicted in FIG. 1B and referenced with reference 1002. Here, the retainment period is one minute. Alternatively, said retainment period may be at least one minute, such as for example two minutes or four minutes.

For said respective cycle, (iii) the controller 14 is also configured to control the at least one valve 15, 16, to release the air volume 2 from the plenum 10 at the end of said retainment period 5. More specifically: The controller 14 controls the second valve 16 to the open position. This is depicted in FIG. 1B and referenced with reference 1003. Optionally, the controller 14 may control the first valve 15 to remain in the closed position, or alternatively in the open position. In embodiments, the controller may also control the flow generator to force the air out of the plenum, so as to facilitate the release of the air volume in the space even further.

In other examples, where no valves are present in the disinfection device, the flow generator may release the air volume from the plenum at the end of the retainment period by forcing (or inducing a flow to flow) the air volume currently retained in the plenum out of the plenum.

Still referring to FIG. 1A and FIG. 1B, the controller 14 controls the light source 11 to emit the disinfecting light 19 during the retainment period of said cycle. Said disinfecting light 19 thereby disinfecting the air volume 2 retained in the plenum 10. Hence, the disinfecting light 19 is emitted when the air volume 2 is retained in the plenum 10.

The disinfecting light 19 comprises a light intensity. Yet even further, in the present embodiment, the controller 14 is also configured to control the light intensity based on the duration of said retainment period. Namely, as the retainment period is one minute in the present embodiment, the light intensity of the emitted disinfecting light 19, in said respective cycle, is 50% of the maximum light intensity of the light source 11. Moreover, in the present embodiment, the controller 14 controls the light source to emit disinfecting light comprising a light spectrum comprising UV-C light.

Other examples may be envisioned similarly. For example, in alternative examples, the retainment period may be two minutes, while the light intensity may be set to 25% of the maximum light intensity of the light source. Yet alternatively, the controller may also be configured to set said light spectrum based on the duration of said retainment period. For example, when the retainment period is two minutes, the light spectrum of the disinfecting light may be UV-A light instead of UV-C light.

In embodiments, the disinfecting light may comprise a first disinfecting light and a different second disinfecting light. The controller may thereby be configured to control the light source to emit the first disinfecting light for a first subperiod during the retainment period of each respective cycle, and the second disinfecting light for a second subperiod during the retainment period of each respective cycle, so as to disinfect the air volume retained in the plenum. The first subperiod thereby precedes the second subperiod. In such an alternative operation of the light source, the first disinfecting light may damage or weaken the pathogens to be disinfected in the air volume, which may be performed with a less hazardous light spectrum, such as 222 nm UVC light, whereas the second disinfecting light may therefore more easily ‘kill’ the genetic matter of the pathogen, with e.g. more hazardous 254 nm UVC light, but thereby requiring less dose of said hazardous 254 nm UVC light.

Still referring to FIG. 1A and FIG. 1B, the retainment period is prestored within the controller 14. For example, the retainment period may be a setting stored in a local memory of the controller 14. Therefore, in the present embodiment, the controller 14 is configured to determine said retainment period based on the predetermined retainment period stored within said controller 14.

Alternatively, the controller may receive an input indicative of said retainment period. Said input may for example be a user input. The controller may then be configured to determine said retainment period based on the received input.

Yet further, in embodiments, the second conduit 161 may be arranged for releasing the air volume to the space with a particular flow behavior or flow pattern. Said second conduit 161 may comprise a nozzle configured to release the air volume in a vortex flow, for such nozzles be known in the art. Such vortex flow may for example be advantageous to distribute disinfected air better in the space, especially when operating the disinfection device in a pulsed or periodic operation. Alternatively, the nozzle may be configured to release the air volume in a plug flow or jet flow may for example be advantageous to distribute disinfected air further in distance in the space.

All in all, the disinfection device 100 depicted in FIG. 1A and FIG. 1B is advantageous, because the disinfection of the air volume 2 of the space 1 occurs intermittently in a plurality of cycles, wherein each respective air volume 2 can be disinfected during a particular retainment period with said disinfecting light 19. As the air volume 2 is retained for the retainment period, and thereby exposed a longer time to the disinfecting light 19, the light source 11 may more effectively disinfect the air volume 2. This improves the disinfection effectiveness of the disinfection device 100.

Furthermore, since the air volume 2 is retained for the retainment period, and thereby exposed longer to the disinfecting light 19, the light source 11 operates the disinfecting light 19 at a lower intensity (i.e. 50% of the maximum intensity), but still ensure the disinfection effectiveness and a germicidal effect. Thereby, the disinfection device 100 requires less power consumption for disinfection, but still maintain a desired disinfection effectiveness (for example compared to light-based disinfection devices in which the air is flowing continuously and at higher flow rate past the disinfecting light source).

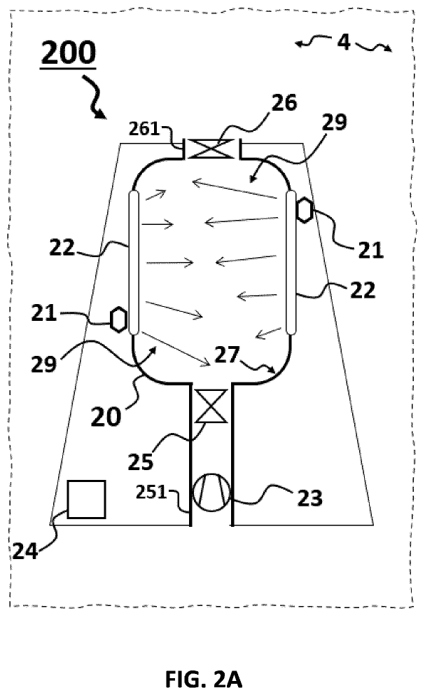

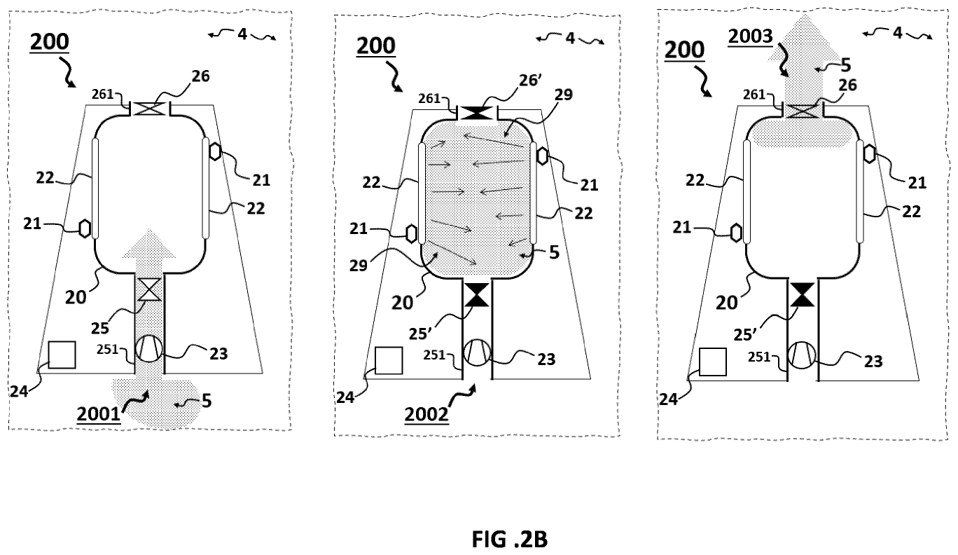

FIG. 2A depicts schematically, by non-limiting example, an embodiment of a disinfection device 200 according to the invention. FIG. 2B depicts schematically, by non-limiting example, the operation of said disinfection device 200. The disinfection device 200 is located within a space 4. Hence, he disinfection device 200 is configured to operate in a plurality of cycles. For each cycle of said plurality of cycles, a respective air volume 5 of said space 4 is disinfected. Thus, the air volume 5 may be a part of the atmosphere of said space 4.

The disinfection device 200 comprises a plenum 20. The plenum 20 is configured to retain the respective air volume 5 to be disinfected. The plenum 20 comprises at least one valve 25, 26. Here, the plenum 20 comprises a first valve 25 and a second valve 26. The plenum 20 optionally comprises a first conduit 251 to the space 4, and a second conduit 261 to the space 4, wherein the first valve 25 is arranged in the first conduit 251 and the second valve 26 is arranged in the second conduit 261. Alternatively, the plenum may be envisioned to comprise a plurality of valves, and/or a plurality of corresponding conduits.

More specifically, the at least one valve 25, 26 is configured to operate between an open position and a closed position. In the open position, the respective valve of the at least one valve is in fluid communication with the space 4. In the closed position, the respective valve of the at least one valve 25, 26 is not in fluid communication with the space 4. Hence, each respective valve 25, 26 is configured to open or close their corresponding conduit 251, 261, to thereby allow or prevent airflow between the plenum 20 and the space 5. The closed position of a valve is referenced in the figures with the reference numeral having an accent (′).

Still referring to FIG. 2A and FIG. 2B, the disinfection device 200 according to the invention comprises a light source 21. The light source 21 is an array of LED light sources configured to emit, in operation, disinfecting light 29. Alternatively, the light source may be a conventional light source, such as a Xenon lamp emitting in the ultraviolet spectrum. The light source 21 is arranged outside the plenum 20. The plenum 20 comprises a plenum wall 27 enclosing the plenum 20. The plenum wall 27 comprises a light guide 22. The light guide 22 is configured to convey light into the plenum 20 for illuminating the air volume 5 in the plenum 20. More specifically, the light source 21 couples the disinfecting light into said light guide 22 for illuminating the plenum 20. Here, the disinfecting light 29 comprises a light spectrum comprising at least one dominant peak wavelength in the ultraviolet-C wavelength range. Alternatively, said disinfecting light may comprise, violet light, Infrared light, UV-A, and/or UV-B light.

The plenum wall may optionally comprise a reflective surface facing into the plenum for reflecting disinfecting light. Yet in further aspects, said light guide may for example cover the whole circumference of said plenum, or cover the whole height of said circumference. Said light guide may enable a larger light output area. For example, the light guide may comprise a light output surface, which light output surface comprises a surface area at least a quarter of the whole surface area of said plenum wall. Yet alternatively, said plenum wall may be transparent for the disinfecting light, such that the externally arranged light source may convey the disinfecting light through the plenum wall into the plenum.

Still referring to FIG. 2A and FIG. 2B, the disinfection device 200 further comprises a flow arrangement (not explicitly referenced to) comprising a flow generator 23. The flow arrangement (at least partly) controls the flow of air through the disinfection device 200. Here, the flow generator 23 is a bellows that is consequently arranged in the first conduit 251. The flow generator 23 is configured to force, in operation, an air volume 5 into the plenum 20. Even further, the flow generator 23 (i.e. the bellows) is configured to pressurize the air volume 5 in the plenum 20. Alternatively, the flow generator may be a fan, ventilator, or other fluid forcing means.

Still referring to FIG. 2A and FIG. 2B, the disinfection device 200 further comprises controller 24. The controller 24 is configured to determine a retainment period. The retainment period is prestored within the controller 14. Alternatively, the controller may receive an input indicative of said retainment period. Said input may for example be a user input, for example a button-press indicative of a duration for the retainment period. The controller may then be configured to determine said retainment period based on the received input.

The controller 24 is configured to the disinfection device 200 for a plurality of repetitive cycles. The controller 24 is configured, for each cycle of said plurality of repetitive cycles, to at least perform the below actions (i), (ii), and (iii).

Namely, for said respective cycle, (i) the controller 24 is configured to control the flow generator 23 to force an air volume 5 into the plenum 20, more specifically to pressurize the air volume in the plenum 20 (with an overpressure). The air volume 5 is a part of the atmosphere of the space 4. Hence, the air volume 5 is drawn in, by the flow generator 23, through the first conduit 251, and through the first valve 25 into the plenum 20. The first valve 25 is thereby automatically operated, as the first valve 25 allows the air volume 5 to flow into the plenum 20 but not in the opposite direction out. The flow generator 23 forcing and pressurizing the air volume 5 into the plenum is depicted in FIG. 2B and referenced with reference 2001. The second valve 26 is controlled to remain closed during the flow generator 23 forcing the air volume 5 into the plenum 20.

For said respective cycle, (ii) the controller 24 is also configured to control the at least one valve 25, 26 to retain the air volume 5 in the plenum 20 for the retainment period. More specifically, the controller 24 controls the first valve 25 in the closed position and the second valve 26 to remain in the closed position. This retaining is depicted in FIG. 1B and referenced with reference 2002. Here, the retainment period is three minutes. Alternatively, said retainment period may be at least one minute, such as for example two minutes or four minutes.

For said respective cycle, (iii) the controller 24 is also configured to control the at least one valve 25, 26, to release the air volume 5 from the plenum 20 at the end of said retainment period. More specifically: The controller 24 controls the second valve 26 to the open position. The first valve 25 remains in the closed position. This is depicted in FIG. 2B and referenced with reference 2003. Since the air volume 5 is pressurized in the plenum 20 during the retainment period, the air volume will be forced out by the pressure difference with the atmosphere of the space 4 at the end of the retainment period when the second valve 26 is brought to the open position.

Still referring to FIG. 2A and FIG. 2B, the controller 24 controls the light source 21 to emit the disinfecting light 29 during the retainment period of said cycle. Said disinfecting light 29 thereby disinfecting, via the light guide 22, the air volume 5 retained in the plenum 20. Hence, the disinfecting light 29 is emitted when the air volume 5 is retained in the plenum 20.

The disinfecting light 29 comprises a light intensity. Yet even further, in the present embodiment, the controller 24 is also configured to control the light intensity based on the duration of said retainment period. Namely, as the retainment period is three minutes in the present embodiment, the light intensity of the emitted disinfecting light 29, in said respective cycle, is 10% of the maximum light intensity of the light source 21.

Other examples may be envisioned similarly. For example, in alternative examples, the controller may determine a retainment period of one minute. For a retainment period of one minute, the light intensity may be set to 50% of the maximum light intensity of the light source. Yet alternatively, the controller may also be configured to set said light spectrum based on the duration of said retainment period. For example, when the retainment period is two minutes, the light spectrum of the disinfecting light may be UV-B light or Xenon light instead of UV-C light.

Yet further, in embodiments, the second conduit 261 may be arranged for releasing the air volume to the space with a particular flow behavior or flow pattern. Said second conduit 161 may comprise a nozzle configured to release the air volume in a vortex flow, for such nozzles be known in the art. Such vortex flow may for example be advantageous to distribute disinfected air better in the space, especially when operating the disinfection device in a pulsed or periodic operation. Alternatively, the nozzle may be configured to release the air volume in a plug flow or jet flow may for example be advantageous to distribute disinfected air further in distance in the space.

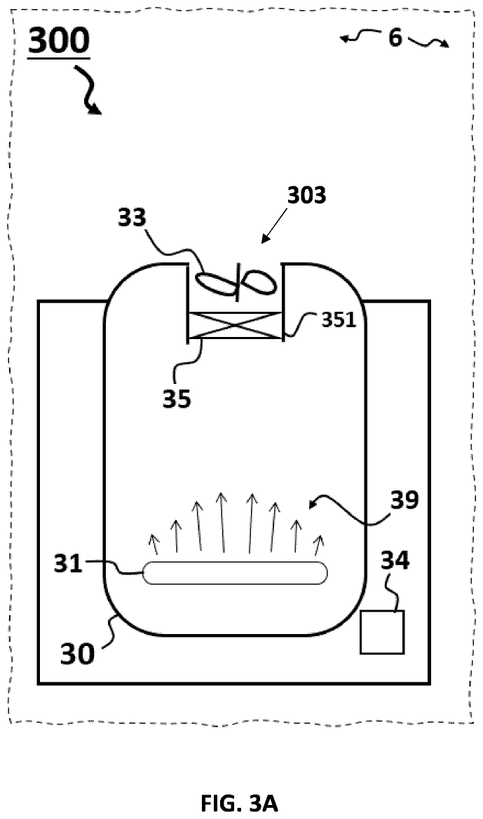

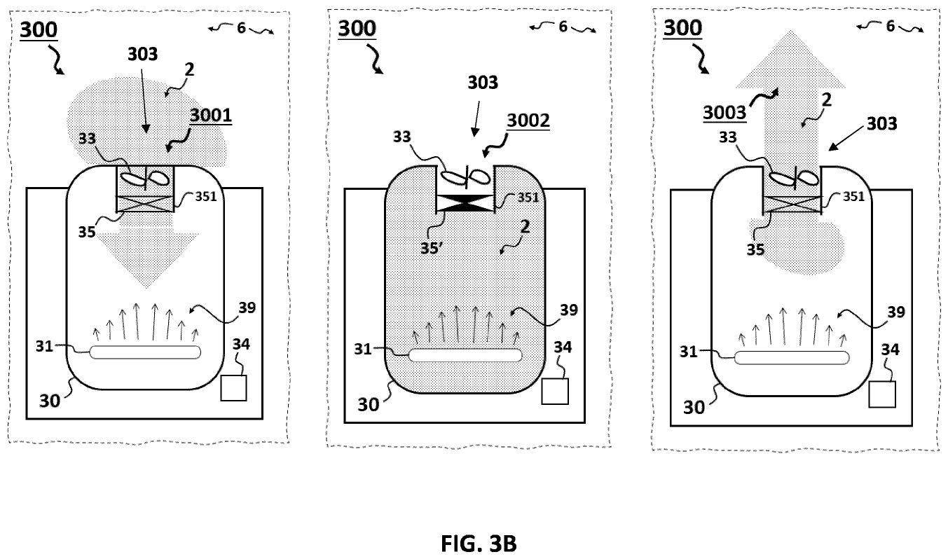

FIG. 3A depicts schematically, by non-limiting example, an embodiment of a disinfection device 300 according to the invention. FIG. 3B depicts schematically, by non-limiting example, the operation of said disinfection device 300. The disinfection device 300 is located within a space 6. Hence, he disinfection device 300 is configured to operate in a plurality of cycles. For each cycle of said plurality of cycles, a respective air volume 7 of said space 6 is disinfected. Thus, the air volume 7 may be a part of the atmosphere of said space 6.

The disinfection device 300 comprises a flow arrangement 303. The flow arrangement 303 comprises, here, a flow generator 35. The flow arrangement 303 may also comprise at least one valve 35, which is embodied as part of the plenum 30.

The disinfection device 300 comprises a plenum 30. The plenum 30 is configured to retain the respective air volume 7 to be disinfected. The plenum 30 optionally comprises said at least one valve 35 of the flow arrangement. Hence, here, the plenum 30 comprises a single valve 35. The valve 35 is arranged within an aperture 351 or conduit of the plenum 30 to the space 6. Alternatively, the plenum may be envisioned to comprise no valves but only said conduits, or a plurality of valves, and/or a plurality of corresponding conduits in which the respective valves are arranged. More specifically, the valve 35 is configured to operate between an open position and a closed position. In the open position, the valve 35 is in fluid communication with the space 6. In the closed position, the valve 35 is not in fluid communication with the space 6. The closed position of a valve is referenced in the figures with the reference numeral having an accent (′).

Still referring to FIG. 3A and FIG. 3B, the disinfection device 300 according to the invention comprises a light source 31. Here, the light source 31 is an Xenon lamp, that is arranged for outputting disinfecting light comprising an ultraviolet light spectrum. The light source 31 emits, in operation, disinfecting light 39 for illuminating the plenum 30. Here, the light source 31 is arranged inside the plenum 30.

More specifically, in this particular embodiment, by non-limiting example, the plenum 30 comprises an upper halve region and a nonoverlapping lower halve region, the upper halve region and the lower halve region comprising a same volume value, wherein the lower volume region is located below the upper volume region with respect to the gravitational direction. The light source 31 is thereby arranged inside the plenum 30 within the lower halve region. When the light source 31 (i.e. the Xenon lamp) is in operation, the light source 31 will also generate heat next to the disinfecting light 39. Said heat will cause buoyancy effects and natural convection of an air volume inside the plenum 30. This will facilitate the mixing of the air volume, and thereby improve the disinfection performance of the disinfection device.

In alternative embodiments, said Xenon lamp may be a UV LED lamp, or a UV Batten, such as UVC LED lamp or UVC batten. In alternative aspects, the plenum wall may optionally comprise a reflective surface facing into the plenum for reflecting the disinfecting light. Yet alternatively, said plenum wall may at least partly be transparent for the disinfecting light, wherein the light source may be arranged outside the plenum.

Still referring to FIG. 3A and FIG. 3B, the disinfection device 200 further comprises a flow generator 33. Here, the flow generator 33 is a ventilator (or: fan). The flow generator 33 is arranged in said aperture 351 or conduit in which also the valve 35 is present (though not necessary to be in the same conduit). The flow generator 33 is configured to force, in operation, an air volume 7 into the plenum 30. Alternatively, said flow generator may e.g. a bellows. Alternatively, the flow generator may be configured to pressurize the air volume in the plenum.

Still referring to FIG. 3A and FIG. 3B, the disinfection device 200 further comprises controller 34. The controller 24 is configured to determine a retainment period.

The retainment period is received by the controller 34 by the controller 34 receiving an input (or: signal) indicative of said retainment period. Alternatively, the controller may determine the retainment period based on a predetermined retainment period (or setting) stored in said controller, or an associated memory.

The controller 34 is configured to control the disinfection device 300, and in particular the flow arrangement 303 thereof, for a plurality of repetitive cycles. The controller 34 is configured, for each cycle of said plurality of repetitive cycles, to at least perform the below actions (i), (ii), and (iii).

Namely, for said respective cycle, (i) the controller 34 is configured to control the flow arrangement 303, in particular the flow generator 33 thereof to force an air volume 7 into the plenum 30. Alternatively, the controller may additionally be configured to control the flow generator to pressurize the air volume in the plenum. The air volume 7 is a part of the atmosphere of the space 6. Hence, the air volume 7 is drawn in, by the flow generator 33, through the valve 35 into the plenum 30. The flow generator 33 forcing the air volume 7 into the plenum is depicted in FIG. 3B and referenced with reference 3001.

For said respective cycle, (ii) the controller 34 is also configured to control the valve 35 to retain the air volume 7 in the plenum 30 for the retainment period. Alternatively, the valve 35 may be a passive valve, that is mechanically able to move in an open and closed position depending on the flow direction through the conduit 351, which flow direction is caused by the operation of the flow generator 33. More specifically, in the present embodiment, the controller 34 controls the valve 35 to be in the closed position. This retaining is depicted in FIG. 3B and referenced with reference 3002. Here, the retainment period is five minutes. Alternatively, said retainment period may be at least one minute, such as for example two minutes or four minutes, or at most ten minutes.

For said respective cycle, (iii) the controller 34 is also configured to control the valve 35 to release the air volume 7 from the plenum 30 at the end of said retainment period. More specifically: The controller 34 controls valve 35 to the open position. This is depicted in FIG. 3B and referenced with reference 3003. Even further, in the present embodiment, the controller 34 is configured to control the flow generator 33 to force the air volume 7 out of the plenum 30 at the end of said retainment period. Hence, the air volume 7 enters and leaves the plenum 30, during operation and in this particular embodiment, through a single (and same) aperture 351 or conduit.

Still referring to FIG. 3A and FIG. 3B, the controller 34 controls the light source 31 to emit the disinfecting light 39 during the retainment period of said cycle. Said disinfecting light 39 is thereby disinfecting, via the light guide 32, the air volume 7 retained in the plenum 30. Hence, the disinfecting light 39 is emitted when the air volume 7 is retained in the plenum 30.

The disinfecting light 39 comprises a light intensity and a spectrum. Yet even further, in the present embodiment, the controller 34 is also configured to control the light intensity and the spectrum based on the duration of said determined retainment period. Namely, as the retainment period is determined as five minutes in the present embodiment, the light intensity of the emitted disinfecting light 39, in said respective cycle, is 5% of the maximum light intensity of the light source 31 and the spectrum is 222 nm UVC. Other examples may be envisioned similarly. For example, in alternative examples, the controller may determine a retainment period of one minute. For a retainment period of one minute, the light intensity may be set to 50% of the maximum light intensity of the light source and to 354 nm UVC.

In an embodiment, the controller is configured to control light source to emit at least one pulse of disinfecting light during the retainment period of each respective cycle, so as to disinfect the air volume retained in the plenum. Each respective pulse of the at least one pulse comprises a time duration between one microsecond and ten milliseconds, for example a tenth of a millisecond.

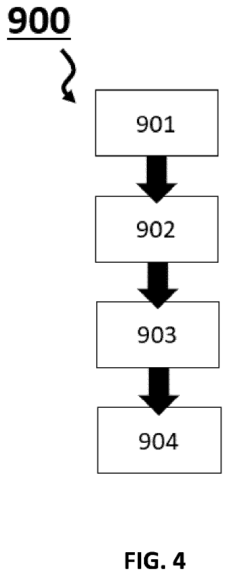

FIG. 4 depicts schematically, by non-limiting example, a method 900 of disinfecting an air volume of a space. The method 900 may be performed by the disinfection devices as depicted in the embodiments of FIGS. 1 to 3. The method comprises, for each cycle of a plurality of repetitive cycles, a step 901 of forcing the air volume of the space into a plenum, a step 902 of retaining the air volume in the plenum for a retainment period, a step 903 of controlling a light source to emit a disinfecting light during the retainment period, so as to disinfect the air volume retained in the plenum, and a step 904 of releasing the air volume from the plenum at the end of said retainment period.

1. A disinfection device for disinfecting an air volume of a space, wherein the disinfection device comprises:
a plenum for retaining the air volume;
a light source for illuminating the plenum;
a flow arrangement;
a controller;
wherein the controller is configured to control, for each cycle of a plurality of repetitive cycles, the flow arrangement to:
(i) force the air volume into the plenum,
(ii) retain the air volume in the plenum for a retainment period,
(iii) release the air volume from the plenum at the end of said retainment period;
wherein the controller is configured to control the light source to emit a disinfecting light during the retainment period of each respective cycle, so as to disinfect the air volume retained in the plenum
wherein the emitted disinfecting light comprises a light intensity; wherein the controller is configured to set said light intensity based on the duration of said retainment period;
wherein the controller is configured to determine said retainment period based on a predetermined retainment period stored within said controller; OR
receive an input indicative of said retainment period, wherein the controller is configured to determine said retainment period based on the received input.

2. The disinfection device according to claim 1, wherein the retainment period is at least one minute.

3. The disinfection device according to claim 1, wherein the emitted disinfecting light comprises a light spectrum;
wherein the controller is configured to set said light spectrum based on the duration of said retainment period.

4. The disinfection device according to claim 1, wherein the emitted disinfecting light comprises a light spectrum;
wherein the light spectrum comprises at least one dominant peak wavelength in the ultraviolet wavelength range from 100-380 nm.

5. The disinfection device according to claim 1, wherein the light source is at least one LED light source.

6. The disinfection device according to claim 1, wherein the light source is at least one Xenon lamp.

7. The disinfection device according to claim 1, wherein the controller is configured to control light source to emit at least one pulse of disinfecting light during the retainment period of each respective cycle, so as to disinfect the air volume retained in the plenum;
wherein each respective pulse of the at least one pulse comprises a time duration between one microsecond and ten milliseconds.

8. The disinfection device according to claim 1, wherein the disinfecting light comprises a first disinfecting light and a different second disinfecting light;
wherein the controller is configured to control the light source to emit the first disinfecting light for a first subperiod during the retainment period of each respective cycle, and the second disinfecting light for a second subperiod during the retainment period of each respective cycle, so as to disinfect the air volume retained in the plenum;
wherein the first subperiod precedes the second subperiod.

9. The disinfection device according to claim 8, wherein the first disinfecting light comprises Far-UVC light;
wherein the second disinfecting light comprises Near-UVC light, preferably 254 nm Near-UVC light.

10. The disinfection device according to claim 1, wherein the plenum comprises a plenum wall enclosing the plenum;
wherein the plenum wall comprises a reflective surface facing into the plenum, wherein the reflective surface is configured to reflect the disinfecting light.

11. The disinfection device according to claim 1, wherein the light source is arranged inside the plenum.

12. The disinfection device according to claim 10, wherein the plenum comprises an upper halve region and a nonoverlapping lower halve region, the upper halve region and the lower halve region comprising a same volume value, wherein the lower volume region is located below the upper volume region with respect to the gravitational direction;
wherein the light source is arranged inside the plenum within the lower volume region.

13. A method of disinfecting an air volume of a space, wherein the method comprises, for each cycle of a plurality of repetitive cycles:
forcing the air volume of the space into a plenum;
retaining the air volume in the plenum for a retainment period;
controlling a light source to emit a disinfecting light during the retainment period, so as to disinfect the air volume retained in the plenum;
releasing the air volume from the plenum at the end of said retainment period.
wherein the emitted disinfecting light comprises a light intensity set based on the duration of said retainment period;
wherein said retainment period is based on a predetermined retainment period stored within a controller; OR said retainment period is based on a received input indicative of said retainment period.
